(19) United States
(12) Patent Application Publication
(10) Pub. No.: US 2022/0406403 A1
(43) Pub. Date: Dec. 22, 2022

(54) SYSTEM AND METHOD FOR GENERATING A NOVEL MOLECULAR STRUCTURE USING A PROTEIN STRUCTURE

(71) Applicant: Innoplexus AG, Eschborn (DE)

(72) Inventors: Vivek Singh, Pune (IN); Ashwin Rathod, Mahur (IN); Bibhash Chandra Mitra, Burdwan (IN); Rohit Yadav, Indore (IN)

(73) Assignee: Innoplexus AG, Eschborn (DE)

(21) Appl. No.: 17/351,317

(22) Filed: Jun. 18, 2021

(51) Int. Cl. G16B 15/30; G16B 5/20; G16B 40/00; G06N 3/08; G06F 30/27

(52) U.S. Cl. CPC G16B 15/30; G16B 5/20; G16B 40/00; G06N 3/08; G06F 30/27

(57) ABSTRACT

A system for generating a novel molecular structure using a protein structure is disclosed. One or more processors generate a protein voxel representation of a protein structure that includes a multichannel three-dimensional (3D) grid that includes a plurality of channels. A cavity region is detected in the protein voxel representation based on a combination of rule-based detection and a deep learning based model. A cavity voxel representation of the cavity region is generated based on upscaling of a regional voxel of the detected cavity region. A ligand voxel representation of a ligand structure is generated based on the cavity voxel representation. A 3D voxel descriptor is determined for a protein-ligand complex based on the protein voxel representation and the ligand voxel representation. A simplified molecular-input line-entry system (SMILES) of a novel molecular structure is generated using a rich 3D embedding vector, which is based on the 3D voxel descriptor.

### FIELD OF TECHNOLOGY

Certain embodiments of the disclosure relate to a method and system for generating a molecular structure. More specifically, certain embodiments of the disclosure relate to a method and system for generating a novel molecular structure using a protein structure.

### BACKGROUND

In the fields of medicine, biotechnology, and pharmacology, drug discovery is the process by which drugs are discovered and/or designed. With recent advancements, computer-aided drug discovery and design methods are utilizing chemical biology and computational drug design approaches for identifying, developing, and optimizing therapeutically important molecular structures. Such computer-aided drug discovery and design methods require various cycles of design, synthesis, characterization, screening, and assays for therapeutic efficacy to yield a series of chemically related molecular structures. Desirable properties of such molecular structures, such as binding affinity to an intended target protein, are progressively tailored to a specific drug discovery goal. However, designing molecules that can bind to the intended target protein and satisfy drug-like properties (such as solubility, bioavailability, and non-toxicity) is an effort-intensive and time-consuming task. Even with highly intensive efforts and substantial time investment (which is typically in years), the rate of success in the area of getting a desirable molecular structure that succeeds in a drug discovery pipeline is very limited.

To design such molecular structures with desirable properties, various methods are being leveraged. Some of the methods are listed, hereinunder: 1) Survey of scientific literature and patents to identify promising molecules/chemical moieties around which molecules with desirable properties can be designed; 2) Use of chemical knowledge-bases and chemical structure drawing tools for designing of molecules with desirable properties based on the existing knowledge-bases; 3) Performing series of in silico high-throughput assays with various endpoints to predict whether the designed molecules possess the desired characteristics; 4) Performing series of high-throughput biological assays with various endpoints using molecules synthesized around a chemical moiety/substructure of interest; and 5) Performing molecular docking based analysis and/or biological assays with purified proteins to assess the binding of the designed molecules to the intended target protein.

However, the abovementioned methods fail to explore the diverse solution space of possible molecular structures (˜10⁶⁰) for generating a molecular structure with desirable properties due to various limitations. One limitation may be the lack of novelty in molecular structure as the molecules are derived primarily by making small alterations to already existing molecules. Another limitation may be that even if novel molecular structures are created by using desirable substructures of existing molecules, factors, such as stability and ease of synthesis, are compromised. Yet another limitation may be that most of the above methods are data-driven, i.e., require a positive dataset of molecules that show the desired properties as a starting point. Thus, for a given protein, for which such a positive dataset is not known or has just a few molecules, the existing methods won't be able to generate good molecules.

Further limitations and disadvantages of conventional and traditional approaches will become apparent to one of skill in the art through comparison of such systems with some aspects of the present disclosure as set forth in the remainder of the present application with reference to the drawings.

### BRIEF SUMMARY OF THE DISCLOSURE

Systems and/or methods are provided for generating a novel molecular structure using a protein structure, substantially as shown in and/or described in connection with at least one of the figures, as set forth more completely in the claims.

These and other advantages, aspects, and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for generating a novel molecular structure using a protein structure, in accordance with an exemplary embodiment of the disclosure.

FIGS. 2A to 2F illustrate exemplary schematic diagrams of various components of a computing device, in accordance with an exemplary embodiment of the disclosure.

FIGS. 3A to 3D depict flowcharts illustrating exemplary operations for generating a novel molecular structure using a protein structure, in accordance with various exemplary embodiments of the disclosure.

FIG. 4 illustrates an inferential pipeline, described in conjunction with FIGS. 3A and 3B, for generating a novel molecular structure using a protein structure, in accordance with an exemplary embodiment of the disclosure.

FIG. 5 is a conceptual diagram illustrating an example of a hardware implementation for a system employing a processing system for generating a novel molecular structure using a protein structure, in accordance with an exemplary embodiment of the disclosure.

### DETAILED DESCRIPTION OF THE DISCLOSURE

Certain embodiments of the disclosure may be found in a method and system for generating a novel molecular structure using a protein structure. Various embodiments of the disclosure provide a method and system that correspond to a solution for a novel molecular structure generation using deep learning (DL) methodology. The proposed method and system may be configured to be an artificial intelligence (AI)/DL and bioinformatics-based model that leverages three-dimensional (3D) characteristics of a protein structure (and its functional binding site) for generating a molecular structure that is optimized for binding to the protein structure of an intended target protein. The proposed method and system is a generic and efficient solution to learn the 3D properties of the intended target protein and corresponding binding sites, which can, in turn, design or generate a ligand that can bind to the site.

Various features of the method and system have been proposed that facilitate in identification or designing of molecules that can bind to the intended target protein and satisfy drug-like properties with minimal effort, maximal timesaving, and a substantially high rate of success for getting desirable molecules that succeed in a drug discovery pipeline. One feature may be a novel method, referred to as ‘Periodic Gaussian Smoothing’, for augmenting voxels in solving the issues of sparsity in the voxel descriptors. Another feature may be a combination of rule-based cavity detection with a DL-based solution for better cavity detection. Yet another feature may be a 3D voxel descriptor for the protein-ligand complex, referred to as ‘Convolved complex voxel’, which can, in turn, be used to generate rich embeddings, referred to as ‘Convoxel fingerprints’. Yet another feature may be a pipeline to improve the generated voxels based on reward functions like affinity scores, novelty, and the like.

In accordance with various embodiments of the disclosure, a method may be provided for generating a molecular structure using a protein structure. The method may include generating, by one or more processors in a computing device, a protein voxel representation of a protein structure that comprises a multichannel 3D grid. The multichannel 3D grid may include a plurality of channels that comprises information regarding a plurality of properties of the protein structure. The method may further include detecting a cavity region in the protein voxel representation of the protein structure based on a combination of rule-based detection and a deep learning-based model. The method may further include generating a cavity voxel representation of the detected cavity region based on at least an upscaling of a regional voxel of the detected cavity region. The method may further include generating a ligand voxel representation of a ligand structure based on at least the cavity voxel representation of the detected cavity region. The method may further include determining a 3D voxel descriptor for a protein-ligand complex based on the protein voxel representation of the protein structure and the ligand voxel representation of the ligand structure. The method may further include generating a simplified molecular-input line-entry system (SMILES) of a novel molecular structure using a rich 3D embedding vector, which is based on the determined 3D voxel descriptor.

In accordance with an embodiment, the plurality of channels in the multichannel 3D grid may include a protein channel that corresponds to the shape of the protein structure, another channel that corresponds to an electrostatic potential of the protein structure, and remaining channels that correspond to two variations of Lennard-Jones potential for a plurality of atom types. The atom types may include a hydrophobic atom, an aromatic atom, a hydrogen bond acceptor, a hydrogen bond donor, a positive ionizable atom, a negative ionizable atom, a metal atom type, and an excluded volume atom.

In accordance with an embodiment, the method may include augmenting the plurality of channels to resolve sparsity in the protein voxel representation. The sparsity may correspond to zero values of one or more voxels in the protein voxel representation.

In accordance with an embodiment, for the generation of the cavity voxel representation of the detected cavity region, the method may further include generating a higher resolution voxel representation of the detected cavity region based on the upscaling of the regional voxel detected cavity region using an AI upscaling operation. The method may further include inverting voxel values in the generated higher resolution voxel representation. The generation of the cavity voxel representation of the cavity region may be further based on the inversion of the voxel values in the generated higher resolution voxel representation.

In accordance with an embodiment, for the determination of the 3D voxel descriptor for a protein-ligand complex, the method may further include generating a multichannel convolved voxel representation of the ligand structure based on convolution of the protein voxel representation and the ligand voxel representation. The multichannel convolved voxel representation may include a set of channels that comprises information regarding different random orientations of the ligand structure. The method may further include predicting an actual complex voxel representation of the protein structure based on a trained deep learning model. The determination of the 3D voxel descriptor for the protein-ligand complex may be based on the multichannel convolved voxel representation of the ligand structure and the actual complex voxel representation of the protein structure.

In accordance with an embodiment, the method may further include training a variational auto encoder (VAE) using another rich 3D embedding vector based on the actual complex voxel representation of the protein structure. A plurality of reward functions may be optimized using a reinforcement learning module on top of the VAE. The method may further include generating a new 3D voxel descriptor for the protein-ligand complex with intended properties based on the optimized plurality of reward functions.

In accordance with an embodiment, the method may further include generating a new SMILES based on the new 3D voxel descriptor.

In accordance with an embodiment, the plurality of reward functions may include affinity, novelty, and absorption, distribution, metabolism, excretion, and toxicity (ADMET).

In accordance with an embodiment, the generated SMILES may correspond to a line notation for describing the novel molecular structure generated based on the multichannel 3D grid of the protein structure. The novel molecular structure may be described using short American Standard Code for Information Interchange (ASCII) strings.

In accordance with an embodiment, the method may further include generating the rich 3D embedding vector using the determined 3D voxel descriptor. The rich 3D embedding vector may correspond to a single vector of predetermined length representing a protein sequence of the protein structure. The rich 3D embedding vector may be used to predict one or more properties that include at least affinity score and potential bioactivity of the novel molecular structure.

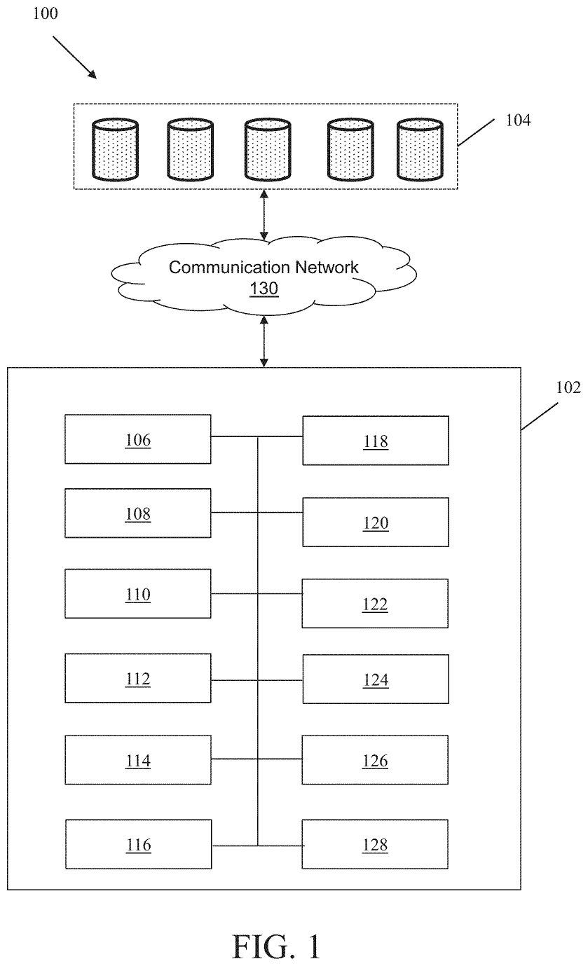

FIG. 1 is a block diagram that illustrates an exemplary system for generating a novel molecular structure using a protein structure, in accordance with an exemplary embodiment of the disclosure. Referring to FIG. 1, a system 100 includes at least a computing device 102 and data sources 104. The computing device 102 comprises one or more processors, such as a voxel generator 106, an augmentation module 108, a cavity detector 110, a 3D generative adversarial network (GAN) 112, a convolved voxel generator 114, a 3D caption generator network 116, a 3D variational autoencoder (VAE) 118, a processor 120, a memory 122, a storage device 124, a wireless transceiver 126, and a user interface 128. The data sources 104 are external or remote resources but communicatively coupled to the computing device 102 via a communication network 130.

In some embodiments of the disclosure, the one or more processors of the computing device 102 may be integrated with each other to form an integrated system. In some embodiments of the disclosure, as shown, the one or more processors may be distinct from each other. Other separation and/or combination of the one or more processors of the exemplary computing device 102 illustrated in FIG. 1 may be done without departing from the spirit and scope of the various embodiments of the disclosure.

The data sources 104 may correspond to a plurality of public resources, such as servers and machines, that may store biomedical knowledge relevant to a specific problem statement and can serve as a starting point for a trainable computational model, for example, a DL-based model. Examples of such data sources 104 may include but are not limited to, ChEMBL database, PubChem, Protein DataBank (PDB), PubMed, Binding DB, SureChEMBL (patent data), and ZINC, known in the art. The data sources 104, such as DUD-E and PDBbind, may include datasets containing protein and ligand complexes and may also be used to train various DL-based models involving voxel generation. For binding site or cavity detection, the data sources 104, such as scPDB and CavBench, may be used.

In accordance with an embodiment, data may be available in a structured format in various public repositories (for example, ChEMBL and PubChem). The structured data may be retrieved from the data sources 104 by various means depending on the data type and size and the options provided by the data source developers. Retrieval mechanisms may include, but not limited to, querying on an online portal, retrieval of data through an FTP server, and retrieval through web services. Moreover, the retrieved data may exist in different forms, including flat files, database collections, and the like. Such retrieved data may require further filtering, which may be performed using parsing scripts and database queries (for example, SQL queries).

In accordance with another embodiment, data may be extracted and derived from unstructured data. An example of deriving datasets from unstructured data may be by constructing a knowledge graph of entities and relationships from the unstructured data. Examples of the unstructured data may include, but are not limited to, research publications, patents, clinical trials, and news. The knowledge graph may be leveraged for creating datasets from the unstructured data based on the entities relevant to the specific problem statement.

Notwithstanding, various types of data sources 104, as exemplified above, should not be construed to be limiting, and various other types of data sources 104 may also be used, without deviation from the scope of the disclosure.

The voxel generator 106 may comprise suitable logic, circuitry, and interfaces that may be configured to execute code that generates a protein voxel representation of a protein structure. The voxel generator 106 may be configured to create good descriptors, i.e., the protein voxel representation, for the given protein structure, which contains information regarding various properties of the protein, such as atom locations and information, bond types, various energies, and charges in a matrix format.

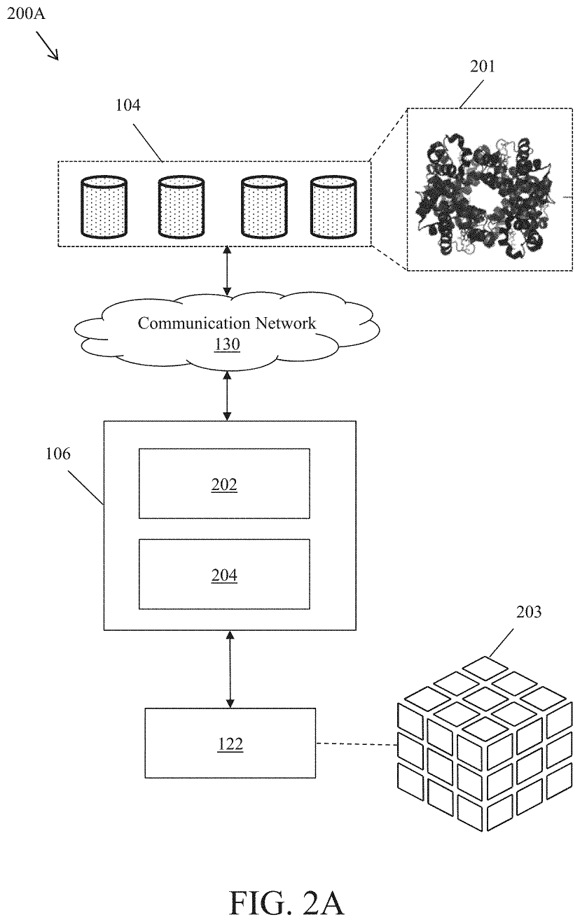

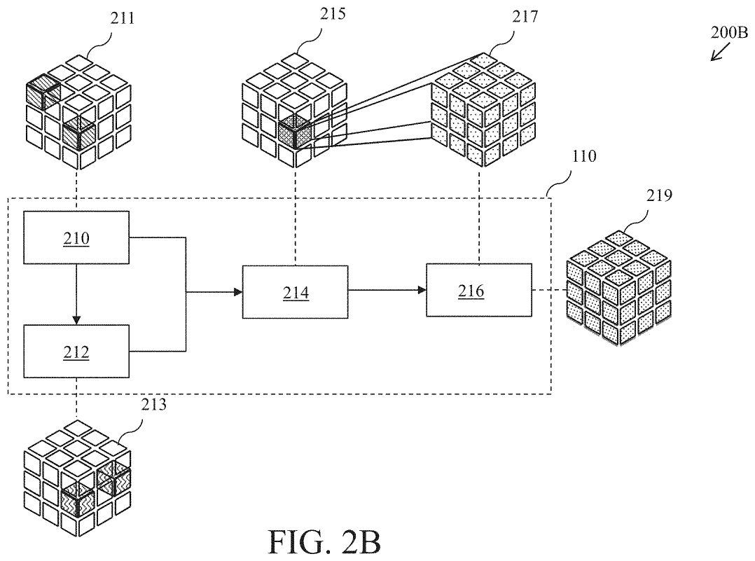

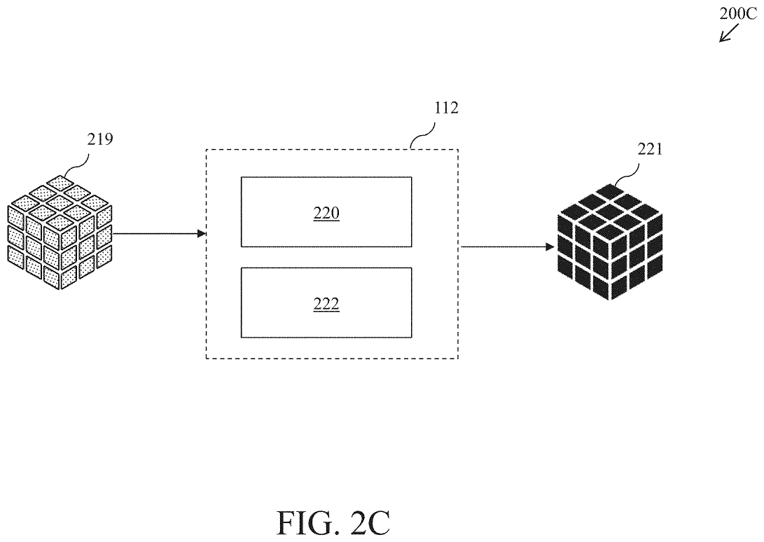

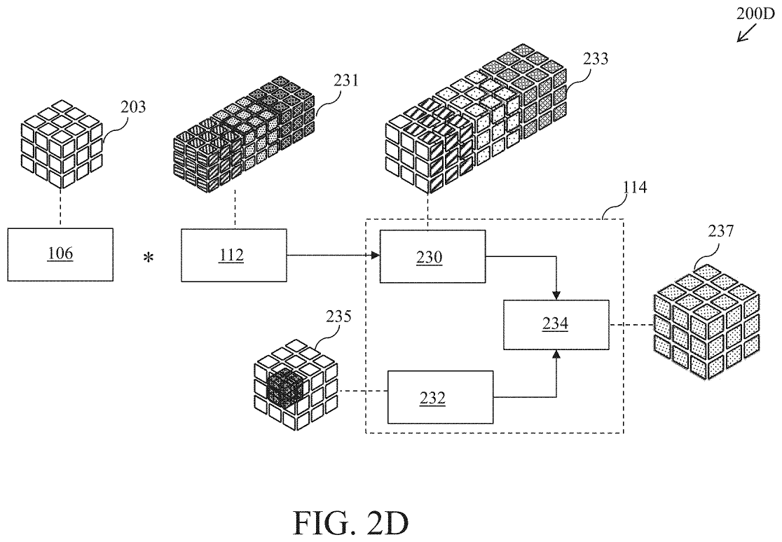

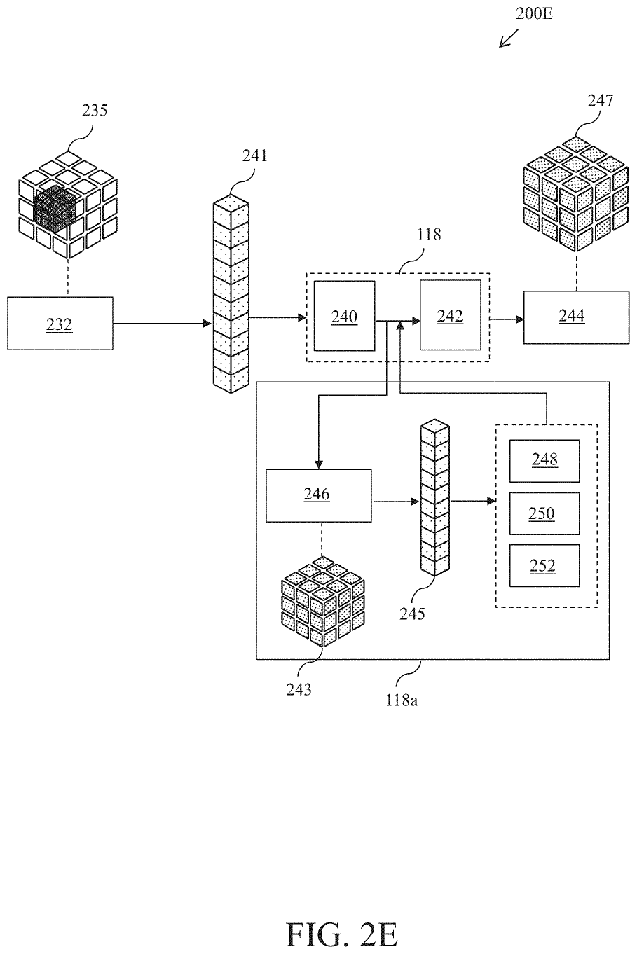

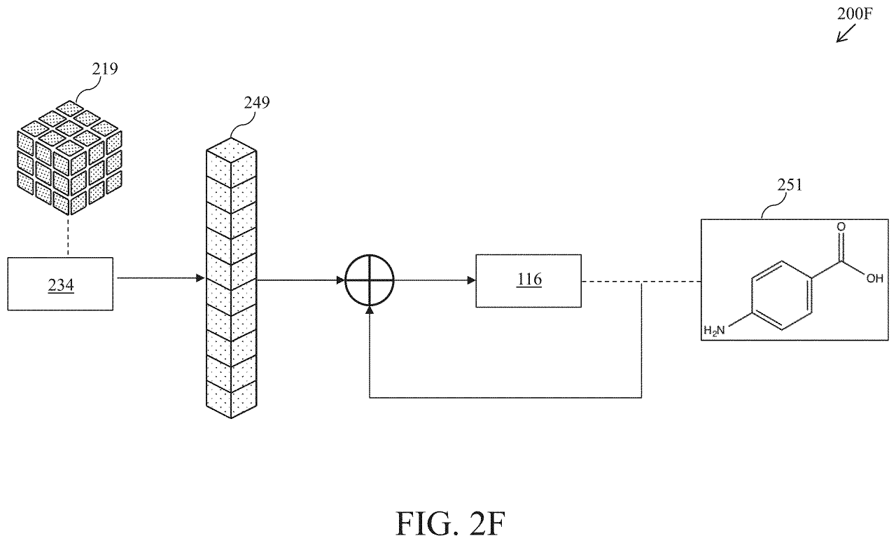

In accordance with an embodiment, by way of an example, the voxel generator 106 may be configured to reading the three-dimensional representation of a macromolecule, such as a given protein structure, from its corresponding Protein Data Bank entry. Atomic coordinates of each atom in the given protein structure may be extracted and stored in a data structure. The voxel generator 106 may be configured to calculate axis-aligned bounding-box enclosing the whole given protein structure by determining minimal and maximal coordinates of each of the atoms in the given protein structure. Based on a desired grid resolution parameter, the voxel generator 106 may be configured to calculate the dimensions of a voxel grid, which will contain the given protein structure. All atomic coordinates previously imported may be translated, scaled, and quantized to the new coordinate system defined by the voxel grid. Each atom center may be mapped in the corresponding voxel in the voxel grid. The voxel generator 106 may be further configured to mark all voxels surrounding a given atom center as occupied by that atom if their distance from its center is less or equal to the corresponding atomic radius. Once all the atoms composing the given protein structure are mapped to the grid, the voxel generator 106 may be configured to generate a protein voxel representation of what is known as the CPK model (also known as the calotte model or space-filling model). In accordance with an embodiment, an exemplary voxel generator is described in FIG. 2A that generates the Van der Waals or the Solvent Accessible surfaces based on extraction of the surface voxels from the protein voxel representation of the CPK volumetric model of the given protein structure. Notwithstanding, the implementation of the voxel generator 106 based on the above examples should not be construed to be limiting, and other methods/means may also be utilized for the implementation without deviating from the scope of the disclosure.

The augmentation module 108 may comprise suitable logic, circuitry, and interfaces that may be configured to execute code that augments the plurality of channels in the multichannel 3D grid to resolve sparsity in the protein voxel representation. The sparsity may correspond to zero values of one or more voxels in the protein voxel representation. The augmentation module 108 may resolve sparsity in the protein voxel representation using a novel method, such as ‘Periodic Gaussian Smoothing (PGS)’. As described above, the channels do contain useful information; however, in certain cases, such channels may be sparse in nature, i.e., mostly filled with zeros due to no potential or atom present in the protein voxel representation. The PGS is a variant of Gaussian smoothing, but instead of convolving with a Gaussian kernel only, a periodic function is added to it, which may cause small perturbations and create small noise. The PGS Kernel may be mathematically expressed as:

$${kernel}_{PGS} = exp ( - \frac{{x}^{2} + {y}^{2} + {z}^{2}}{2 {σ}^{2}} ) sin ( 2 π ( \sqrt{\frac{{x}^{2} + {y}^{2} + {z}^{2}}{{σ}^{2}}} + \frac{1}{4} ) )$$

The cavity detector 110 may comprise suitable logic, circuitry, and interfaces that may be configured to execute code that detects a cavity region in the protein voxel representation of the given protein structure based on a combination of rule-based detection and a deep learning-based model. In accordance with an embodiment, for the generation of the cavity voxel representation of the detected cavity region, the cavity detector 110 may be configured to generate a higher resolution voxel representation of the detected cavity region based on upscaling of regional voxel detected cavity region using an AI upscaling technique. The cavity detector 110 may be further configured to invert voxel values in the generated higher resolution voxel representation.

Specifically, the cavity detector 110 may predict a binding site where a ligand structure should bind in the given protein structure. For determining the binding site, various algorithms, such as LIGSITE, may give the best results based on the geometric properties of the given protein structure. However, there are many other non-geometric factors for consideration while binding, and hence a novel hybrid model using the results above and a deep learning approach is introduced. The scanning results of LIGSITE may be used as a new channel along with the other channels created by the voxel generator 106. Such final voxels may be used to detect the final cavity using an object detection model, such as the Faster Regional CNN (FRCNN)-based object detection model, known in the art.

After detection of the cavity, the cavity detector 110 may be configured to upscale the voxels using AI upscaling techniques, such as Enhanced Super-Resolution Generative Adversarial Networks (ESRGAN), known in the art. Such upscaling may provide the voxel representation of the cavity region of the given protein structure. Thereafter, inversion of the values may be carried out to generate the cavity voxel representation. The inversion may be performed based on the following mathematical expression:

$$\tilde{V} ( x , y , z ) = 1 - \frac{V ( x , y , z )}{max ( V )}$$

Notwithstanding, the implementation of the cavity detector 110 based on the above examples should not be construed to be limiting, and other methods/means may also be utilized for the implementation without deviating from the scope of the disclosure. An exemplary cavity detector is described in FIG. 2B, in accordance with an exemplary embodiment of the disclosure.

The 3D GAN 112 may comprise suitable logic, circuitry, and interfaces that may be configured to execute code that generates a ligand voxel representation of a ligand structure based on at least the cavity voxel representation of the detected cavity region. The 3D GAN 112 may be a multimodal 3D Generative Adversarial Network that may contain two independent neural networks, an encoder, and a generator. The two independent neural networks may be configured to work independently and may act as adversaries. In other words, the 3D GAN 112 contains only two feed-forward mappings, the encoder, and the generator, operating in opposite directions. The encoder may include a classifier that may be trained to perform the task of discriminating among data samples. The generator may generate random data samples that resemble real samples, but which may be generated including, or may be modified to include, features that render them as fake or artificial samples. The neural networks that include the encoder and generator may typically be implemented by multi-layer networks consisting of a plurality of processing layers, for example, dense processing, batch normalization processing, activation processing, input reshaping processing, Gaussian dropout processing, Gaussian noise processing, two-dimensional convolution, and two-dimensional up sampling. Notwithstanding, the implementation of the 3D GAN 112 based on the above examples should not be construed to be limiting, and other methods/means may also be utilized for the implementation without deviating from the scope of the disclosure. An exemplary 3D GAN is described in FIG. 2C, in accordance with an exemplary embodiment of the disclosure.

The convolved voxel generator 114 may comprise suitable logic, circuitry, and interfaces that may be configured to execute code that determines a 3D voxel descriptor for a protein-ligand complex based on the protein voxel representation of the given protein structure and the ligand voxel representation of the ligand structure. In accordance with an embodiment, for the prediction of the 3D voxel descriptor for the protein-ligand complex, the convolved voxel generator 114 may be configured to generate a multichannel convolved voxel representation of the ligand structure based on convolution of the protein voxel representation and the ligand voxel representation. The multichannel convolved voxel representation may include a set of channels that comprises information regarding different random orientations of the ligand structure. The purpose of the model of the convolved voxel generator 114 is not only to learn the physical and chemical properties of a complex but also the geometric attributes of how the ligand structure changes geometrically (in terms of shape, size, rotation, and the like) in order to create the corresponding protein-ligand complex. Thus, random channels corresponding to the random orientations of the ligand structure may be generated at first, and then the model may learn about the other significant orientations that result in the final protein-ligand complex.

In accordance with an embodiment, the convolved voxel generator 114 may be further configured to predict an actual complex voxel representation of the given protein structure based on a trained deep learning model. The actual complex voxel representation may be a voxelized version of PDB structures which may be found in databases, such as BindingDB and NLDB. Such databases contain structures of protein and ligand complexes and may be treated as ground truths. The model of the convolved voxel generator 114, in turn, may be configured to learn to generate or predict such voxels from the given protein structure 201 and the corresponding ligand structure. In such embodiment, the determination of the 3D voxel descriptor for the protein-ligand complex may be based on the multichannel convolved voxel representation of the ligand structure and the actual complex voxel representation of the given protein structure.

In accordance with an embodiment, the ligand voxel representation, as discussed above, may be used to generate the novel 3D voxel descriptor, referred to as ‘convolved complex voxel’. The 3D voxel descriptor may be generated using a model trained to generate the complex voxel using the voxel representations of the ligand and the given protein structure. As the first step, multiple channels are generated for the ligand voxel representation, each of which corresponds to a random orientation of the ligand structure. Such multichannel convolved voxel representation of the ligand structure is then convolved over the given protein structure, and a 3D-CNN model is trained to predict the actual complex voxel representation.

The 3D voxel descriptor, thus generated, may be used to generate a rich 3D embedding vector, referred to as ‘3D convoxel fingerprint’. In general, a 3D embedding vector may correspond to a molecular fingerprint that is a bit string representation of a chemical structure in which each position indicates the presence (1) or absence (0) of chemical features as defined in the design of the fingerprint. Various known in the art molecular fingerprints, such as Morgan, MACCS, and RDK, and DL-based fingerprints, may be generated using certain physiological and structural properties of the molecules. Such fingerprints may be used in various downstream applications, such as ADMET predictor and QSAR known in the art models, but still have multiple limitations and constraints. In accordance with an embodiment of the disclosure, such limitations and constraints are removed as the rich 3D embedding vector is based on not only structural and physicochemical properties but also the protein complex properties. Thus, the rich 3D embedding vector is richer in comparison to other molecular fingerprints. Such rich 3D embedding vector may be used to predict various properties of a complex structure, such as affinity scores, potential bioactivity of ligand (such as K_{D}, IC50 (Inhibitory concentration 50)), and the like.

Notwithstanding, the implementation of the convolved voxel generator 114 based on the above examples should not be construed to be limiting, and other methods/means may also be utilized for the implementation without deviating from the scope of the disclosure. An exemplary convolved voxel generator is described in FIG. 2D, in accordance with an exemplary embodiment of the disclosure.

The 3D caption generator network 116 may comprise suitable logic, circuitry, and interfaces that may be configured to execute code that generates a simplified molecular-input line-entry system (SMILES) using the rich 3D embedding vector, which is based on the predicted 3D voxel descriptor. In accordance with an embodiment, using the rich 3D embedding vector, a 3D caption generator network 116 may be trained to generate the SMILES. The SMILES may correspond to a line notation for describing a novel molecular structure that is generated based on the multichannel 3D grid of the protein structure 201. In accordance with an embodiment, the novel molecular structure may be described using short American Standard Code for Information Interchange (ASCII) strings. Other linear notations may include, for example, the Wiswesser line notation (WLN), ROSDAL, and SYBYL Line Notation (SLN).

In accordance with an embodiment, the model may be based on sequence generation using masked multi-headed attention layers and feed-forward layers, as used in OpenAl's GPT-2, and may be implemented using transformer decoder layers in an open-source machine learning library, such as Pytorch. The SMILES may be generated using the rich 3D embedding vector as the starting of the sequence and keep on decoding till the total number of tokens reaches the padding length. After the generation of all the tokens, inverse tokenization may be carried out to generate the final SMILES. Notwithstanding, the implementation of the 3D caption generator network 116 based on the above examples should not be construed to be limiting, and other methods/means may also be utilized for the implementation without deviating from the scope of the disclosure. An exemplary 3D caption generator network is described in FIG. 2F, in accordance with an exemplary embodiment of the disclosure.

The 3D VAE 118 may comprise suitable logic, circuitry, and interfaces that may be configured to execute code that is trained using another rich 3D embedding vector based on the actual complex voxel representation of the given protein structure to generate a new or improved 3D voxel descriptor. In general, the 3D VAE 118 may be defined as being an autoencoder whose training is regularized to avoid overfitting and ensure that the latent space has good properties that enable the generative process.

On top of the 3D VAE 118, reinforcement learning may be utilized to optimize a plurality of reward functions. The plurality of reward functions may include affinity, novelty, and absorption, distribution, metabolism, excretion, and toxicity (ADMET). Accordingly, the 3D VAE 118 may be configured to generate the new 3D voxel descriptor for the protein-ligand complex with intended properties based on the optimized plurality of reward functions. Notwithstanding, the implementation of the 3D VAE 118 based on the above example should not be construed to be limiting, and other methods/means may also be utilized for the implementation without deviating from the scope of the disclosure. An exemplary 3D VAE is described in FIG. 2E, in accordance with an exemplary embodiment of the disclosure.

The processor 120 may comprise suitable logic, circuitry, interfaces, and/or code that may be operable to process and execute a set of instructions stored in the memory 122 or the storage device 124. In some embodiments, multiple processors and/or multiple buses may be used, as appropriate, along with multiple memories and types of memory. Also, multiple processors, each providing portions of the necessary operations (for example, as a server cluster, a group of servers, or a multi-processor system), may be inter-connected and integrated. The processor 120 may be implemented based on a number of processor technologies known in the art. Examples of the processor may be an X86-based processor, a Reduced Instruction Set Computing (RISC) processor, an Application-Specific Integrated Circuit (ASIC) processor, a Complex Instruction Set Computing (CISC) processor, and/or other processors.

The memory 122 may comprise suitable logic, circuitry, and/or interfaces that may be operable to store a machine code and/or a computer program with at least one code section executable by the processor 120. The memory 122 may be configured to store information within the computing device 102. In some embodiments, the memory 122 may be a volatile memory unit or units. In other embodiments, the memory 122 may be a non-volatile memory unit or units. In yet other embodiments, the memory 122 may be another form of computer-readable medium, such as a magnetic or optical disk. Examples of forms of implementation of the memory 122 may include, but are not limited to, a Random Access Memory (RAM), a Read-Only Memory (ROM), a Hard Disk Drive (HDD), and/or a Secure Digital (SD) card.

The storage device 124 may be capable of providing mass storage for the computing device 102. In some embodiments, the storage device 124 may be or contain a computer-readable medium, such as a hard disk device, an optical disk device, or a tape device, a flash memory or other similar solid-state memory device, or an array of devices, including devices in a storage area network or other configurations. A computer program product may be tangibly embodied in an information carrier. The information carrier may be a computer-readable or machine-readable medium, such as the memory 122 or the storage device 124. The computer program product may also contain instructions that, when executed, perform one or more methods, such as those described in the disclosure.

The wireless transceiver 126 may comprise suitable logic, circuitry, interfaces, and/or code that may be operable to communicate with the other servers and electronic devices via a communication network. The wireless transceiver 126 may implement known technologies to support wired or wireless communication of the computing device 102 with the communication network. The wireless transceiver 126 may include, but is not limited to, an antenna, a radio frequency (RF) transceiver, one or more amplifiers, a tuner, one or more oscillators, a digital signal processor, a coder-decoder (CODEC) chipset, and/or a local buffer. The wireless transceiver 126 may communicate via wireless communication with networks, such as the Internet, an Intranet, and/or a wireless network, such as a cellular telephone network. The wireless communication may use any of a plurality of communication standards, protocols, and technologies, such as a Global System for Mobile Communications (GSM), Enhanced Data GSM Environment (EDGE), wideband code division multiple access (W-CDMA), code division multiple access (CDMA), time division multiple access (TDMA), Long Term Evolution (LTE), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The user interface 128 may comprise suitable logic, circuitry, and interfaces that may be configured to present the results of the 3D VAE 118. The results may be presented in the form of an audible, visual, tactile, or other output to a user, such as a researcher, a scientist, a principal investigator, and a health authority, associated with the computing device 102. As such, the user interface 128 may include, for example, a display, one or more switches, buttons or keys (e.g., a keyboard or other function buttons), a mouse, and/or other input/output mechanisms. In an example embodiment, the user interface 128 may include a plurality of lights, a display, a speaker, a microphone, and/or the like. In some embodiments, the user interface 128 may also provide interface mechanisms that are generated on display for facilitating user interaction. Thus, for example, the user interface 128 may be configured to provide interface consoles, web pages, web portals, drop-down menus, buttons, and/or the like, and components thereof to facilitate user interaction.

The communication network 130 may be any kind of network or a combination of various networks, and it is shown illustrating exemplary communication that may occur between the data sources 104 and the computing device 102. For example, the communication network 130 may comprise one or more of a cable television network, the Internet, a satellite communication network, or a group of interconnected networks (for example, Wide Area Networks or WANs), such as the World Wide Web. Although a communication network 130 is shown, the disclosure is not limited in this regard. Accordingly, other exemplary modes may comprise uni-directional or bi-directional distribution, such as packet-radio and satellite networks.

FIG. 2A illustrates an exemplary schematic diagram 200A of a voxel generator, in accordance with an exemplary embodiment of the disclosure. With reference to FIG. 2A, there is shown an exemplary schematic voxel generator, such as the voxel generator 106, as introduced in FIG. 1, interfaced with the data sources 104 and the memory 122, as shown in FIG. 1. The voxel generator 106 may include a set of interfaces 202 configured to receive structured and unstructured data from the data sources 104. One or more of the data sources 104, such as macromolecular structural data repositories, may store proteins in the form of PDB files, which are a standard way of representing a macromolecular structure. However, proteins in such form, such as given protein structure 201, provide only limited surface representations, primarily aimed for visual purposes. Thus, the given protein structure 201 cannot be used in DL-based models.

The voxel generator 106 may further include one or more modules 204 that may be configured to execute algorithms retrieved from the memory 122 that generate a protein voxel representation 203 of the given protein structure 201. As known, a voxel is the tiniest distinguishable element of a 3D object that represents a single data point on a regularly spaced 3D grid and contains multiple scalar values (vector data). The protein voxel representation 203, as generated by the voxel generator 106, may be a data descriptor that is encoded with biological data in a way that enables the expression of various structural relationships associated with the given protein structure 201. The geometries of the protein voxel representation 203 may be represented using voxels laid out on various topographies, such as 3-D Cartesian/Euclidean space, 3-D non-Euclidean space, manifolds, and the like. For example, the protein voxel representation 203 illustrates a sample 3D grid structure including a series of sub-containers or channels.

In accordance with an embodiment, for each voxel, such as the protein voxel representation 203, a compendium of atomic-based pharmacophoric properties may be defined. Voxel occupancy may be defined with respect to the atoms in the given protein structure 201 depending on corresponding excluded volume and other seven atom properties: hydrophobic, aromatic, hydrogen bond acceptor or donor, positive or negative ionizable, and metallic. In an exemplary scenario, atom types of AutoDock 4, which is a known molecular modeling simulation software, may be used with the pre-specified rules to assign each atom to a specific channel. Non-protein atoms may be filtered out of the calculation. Atom occupancies may be calculated by taking the simplest approximation for the pair correlation function defined by the following mathematical expression:

g(r)=exp(−βV(r))

where V (r)=ϵ(r_{vdw}/r)¹² is the repulsive component of a Lennard-Jones potential and r_{vdw} is the Van der Waals atom radius. For simplicity, the same ϵ is used for each atom type, such that βϵ=1. The single-atom occupancy estimate may be therefore given by the following mathematical expression:

n(r)=1−exp(−(r_{vdw}/r)¹²))

Finally, the occupancy for the protein voxel representation 203 may be calculated as the maximum of the contribution of all atoms belonging to that channel at its center. Accordingly, the voxel generator 106 may be configured to create good descriptors, i.e., the protein voxel representation 203, for the given protein structure 201, which contain information regarding various properties of the protein, such as atom locations and information, bond types, various energies, and charges in a matrix format.

Thus, voxelization of the given protein structure 201 is carried out to convert the given protein structure 201 into the protein voxel representation 203 with multichannel 3D grids. The multichannel 3D grid includes a plurality of channels that comprises information regarding a plurality of properties of the given protein structure 201. For example, a protein channel, such as Channel-1, may correspond to the shape of the given protein structure 201. A set of channels, such as Channels- 2 to 17, may correspond to two variations of Lennard-Jones potential for a plurality of atom types. The atom types may include a hydrophobic atom, an aromatic atom, a hydrogen bond acceptor, a hydrogen bond donor, a positive ionizable atom, a negative ionizable atom, a metal atom type, and an excluded volume atom. Specifically, the Channels- 2 to 9 correspond to Van der Waals energy using the 12-6 L-J equation, and the Channels-10 to 17 correspond to hydrogen bonding energy using the 12-10 L-J equation. Finally, another channel, such as Channel-18, may correspond to an electrostatic potential of the given protein structure 201.

The voxel generator 106 may be configured to export the protein voxel representation 203, which is a voxelized surface, to the memory 122 or the storage device 124. In an example, the protein voxel representation 203 may be exported to the memory 122 in a Point Cloud Data file format of the Point Cloud Library (PCL) because of its simplicity, compatibility, and compactness with different scientific visualization programs. Notwithstanding, other file formats may also be used without deviation from the scope of the disclosure.

FIG. 2B illustrates an exemplary schematic diagram 200B of a cavity detector, in accordance with an exemplary embodiment of the disclosure. With reference to FIG. 2B, there is shown an exemplary schematic cavity detector, such as the cavity detector 110, that includes a rule-based detector 210, a DL-based detector 212, a hybrid cavity detector 214, and an upscaling module 216.

In accordance with an embodiment, the rule-based detector 210 may correspond to a, for example, Geometry and Connolly surface-based method, based on which a molecular representation of the given protein structure 201 is generated. The rule-based detector 210 may generate a first voxel representation 211 based on a prediction of a binding site where a ligand may bind in the given protein structure 201, using geometric properties of the given protein structure 201. In accordance with an embodiment, the rule-based detector 210 may execute LIGSITE program retrieved from the memory 122. The LIGSITE program automatically detects pockets on the surface of a protein structure that may act as binding sites for small molecule ligands.

As, above, the DL-based detector 212 may be configured to generate a second voxel representation 213 based on a prediction of a binding site where a ligand may bind in the given protein structure 201. However, the DL-based detector 212 may predict a binding site where the ligand may bind in the given protein structure 201, based on non-geometric properties of the given protein structure 201.

The hybrid cavity detector 214 may be configured to determine final voxels based on output provided by the rule-based detector 210 and the DL-based detector 212 to predict final voxels corresponding to the binding site in the given protein structure 201. The hybrid cavity detector 214 may use the scanning results of LIGSITE as a new channel along with the other channels created by the voxel generator 106. The hybrid cavity detector 214 may use such final voxels to detect the final cavity using a detection model, for example, Faster Regional CNN (FRCNN) based object detection model and generate a hybrid voxel representation 215.

The upscaling module 216 may be configured to upscale the detected cavity in the hybrid voxel representation 215 using AI to generate a higher resolution voxel representation 217 and invert the voxels, i.e., ones for the zeros and zeros for ones. Such inversion may convert the protein voxel representation 203 to a cavity voxel representation 219.

FIG. 2C illustrates an exemplary schematic diagram 200C of a 3D GAN, in accordance with an exemplary embodiment of the disclosure. With reference to FIG. 2C, there is shown an exemplary schematic 3D GAN, such as the 3D GAN 112, that includes an encoder 220 and a generator 222. The encoder 220 may be configured to receive the cavity voxel representation 219 generated by the cavity detector 110 and return a latent vector as an output. More specifically, the encoder, with learnable parameters, maps the data space of the cavity voxel representation 219 to the latent space. On the other hand, the generator 222, with the learnable parameters, runs in the opposite direction. The generator 222 may be configured to receive the latent vector, generated by the encoder 220, as input and returns a ligand voxel representation 221 as output.

FIG. 2D illustrates an exemplary schematic diagram 200D of a convolved voxel generator, in accordance with an exemplary embodiment of the disclosure. With reference to FIG. 2D, there is shown an exemplary schematic convolved voxel generator model, such as the convolved voxel generator 114, that includes a multichannel voxel generator 230, a CNN model 232, and a complex voxel generator 234, in addition to the voxel generator 106 and the 3D GAN 112, as described in FIG. 1. The voxel generator 106 generates the protein voxel representation 203, and the 3D GAN 112 generates multi-orientated ligand voxel representation 231, which is similar to the ligand voxel representation 221 except for the fact that the multi-orientated ligand voxel representation 231 includes the ligand voxel representation 221 in multiple orientations. The multichannel voxel generator 230 may be configured to convolve the multi-orientated ligand voxel representation 231 over the protein voxel representation 203, and thus, generate a multichannel convolved voxel representation 233 that includes multiple channels for the multi-orientated ligand voxel representation 231, each of which corresponds to a random orientation of the ligand structure. The CNN model 232 may correspond to a 3D-CNN model that is trained to predict an actual complex voxel representation 235. Finally, the complex voxel generator 234 may be configured to generate a novel 3D descriptor, referred to as ‘convolved complex voxel’, using a model trained to generate a 3D voxel descriptor 237 using the protein voxel representation 203 and the multi-orientated ligand voxel representation 231. Specifically, the complex voxel generator 234 may be configured to determine a difference between the multi-orientated ligand voxel representation 231 and the actual complex voxel representation 235, which facilitates the model of the convolved voxel generator 114 to learn and improve itself. Accordingly, the convolved voxel generator 114 may generate and/or predict the 3D voxel descriptor 237 using corresponding protein and ligand voxels, i.e., the multi-orientated ligand voxel representation 231 and the actual complex voxel representation 235.

FIG. 2E illustrates an exemplary schematic diagram 200E of a 3D VAE, in accordance with an exemplary embodiment of the disclosure. With reference to FIG. 2E, there is shown an exemplary schematic 3D VAE, such as the 3D VAE 118, that includes a VAE encoder 240 and a VAE generator 242. In accordance with an additional embodiment, FIG. 2F also illustrates the CNN model 232, a new complex voxel generator 244, and a reinforcement learning module 118a. The reinforcement learning module 118a further comprises a reinforced generator 246, a convolved voxel 243, a second rich 3D embedding vector 245, an affinity predictor 248, a novelty predictor 250, and an ADMET predictor 252.

On the whole, the CNN model 232 may be configured to generate the actual complex voxel representation 235 from which a first rich 3D embedding vector 241 is generated. The 3D VAE 118 may be trained by using the first rich 3D embedding vector 241 to generate a new 3D voxel descriptor 247. Specifically, the VAE encoder 240 may be configured to encode the input, i.e., the first rich 3D embedding vector 241 (generated by the CNN model 232), as a distribution over the latent space. The first rich 3D embedding vector 241 may be encoded as a distribution with some variance instead of a single point, which is enforced to be close to a standard normal distribution. Thereafter, from such distribution, a point from the latent space may be sampled. The sampled output may be transmitted to the reinforcement learning module 118a.

The reinforced generator 246 in the reinforcement learning module 118a may be configured to generate the convolved voxel 243 based on the received sampled output. The convolved voxel 243 is further used to create the second rich 3D embedding vector 245. The second rich 3D embedding vector 245 is used by the affinity predictor 248, the novelty predictor 250, and the ADMET predictor 252 to optimize a plurality of reward functions that are returned to the VAE generator 242. The VAE generator 242, in conjunction with the new complex voxel generator 244, may be configured to generate the new 3D voxel descriptor 247 for the protein-ligand complex with intended properties based on the optimized plurality of reward functions. The plurality of reward functions is carefully designed based on the properties of interest along with the properties which strictly should not be present in the novel molecular structure of the given protein structure 201.

FIG. 2F illustrates an exemplary schematic diagram 200F of a 3D caption generator network, in accordance with an exemplary embodiment of the disclosure. With reference to FIG. 2F, there is shown an exemplary schematic 3D caption generator network, such as the 3D caption generator network 116, that receives a rich 3D embedding vector 249 as an input to generate SMILES of a novel molecular structure 251. The rich 3D embedding vector 249 is based on the predicted convolved complex voxel, i.e., the 3D voxel descriptor 237. In accordance with an embodiment, using the rich 3D embedding vector 249, the 3D caption generator network 116 may be trained to generate the SMILES of the novel molecular structure 251. The model may be based on sequence generation using masked multi-headed attention layers and feed-forward layers, as used in OpenAl's GPT-2, and may be implemented using transformer decoder layers in an open-source machine learning library, such as Pytorch. SMILES of the novel molecular structure 251 may be generated using the rich embedding vector 249 as the starting of the sequence and keep on decoding till the total number of tokens reaches the padding length. After the generation of all the tokens, inverse tokenization may be carried out to generate the final SMILES of the novel molecular structure 251.

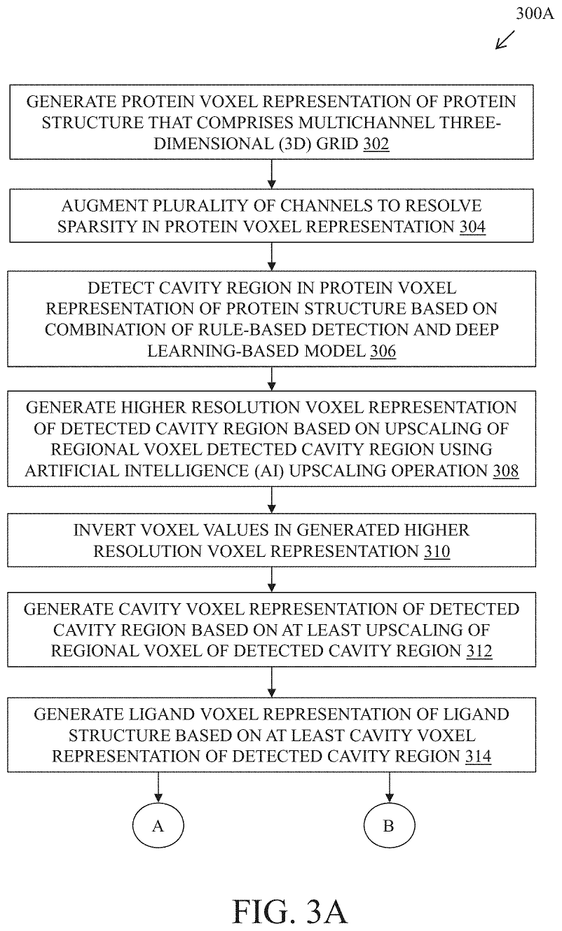

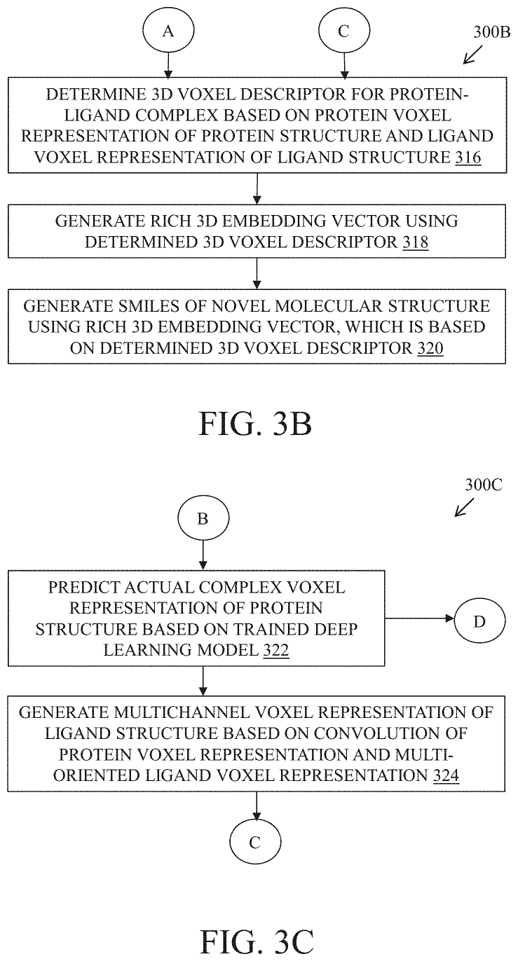

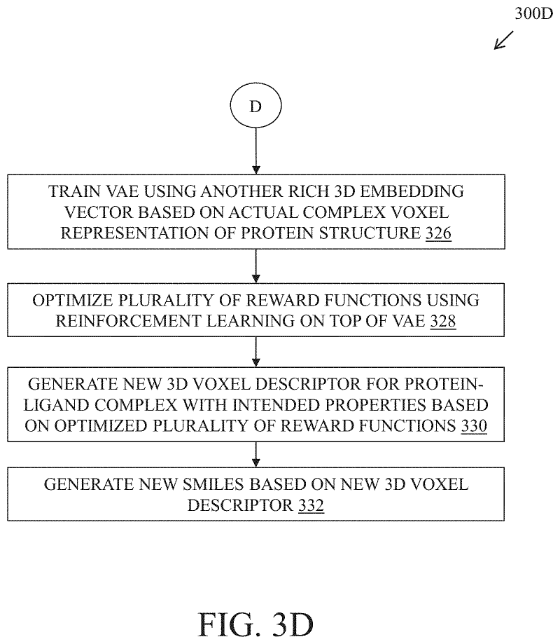

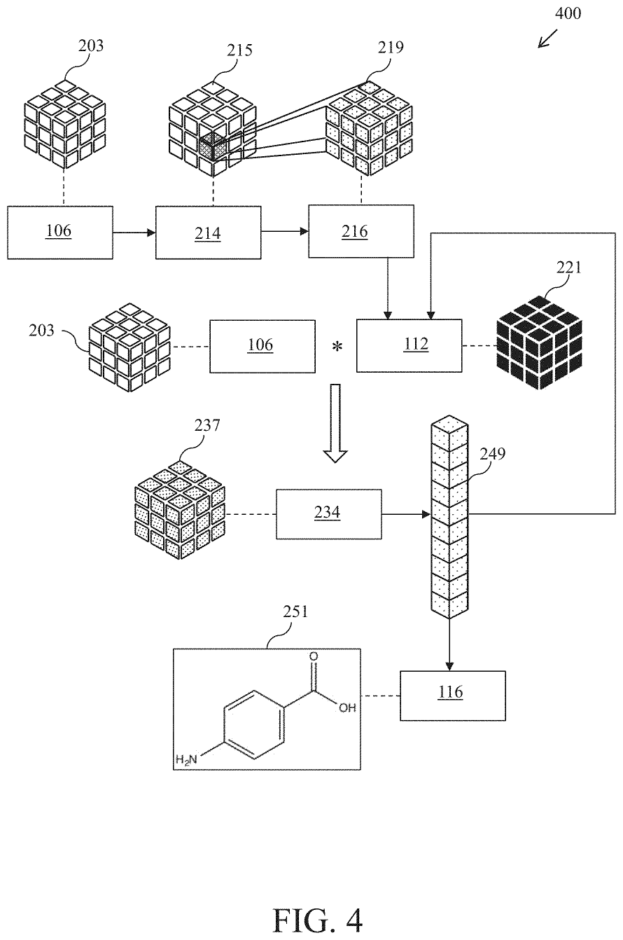

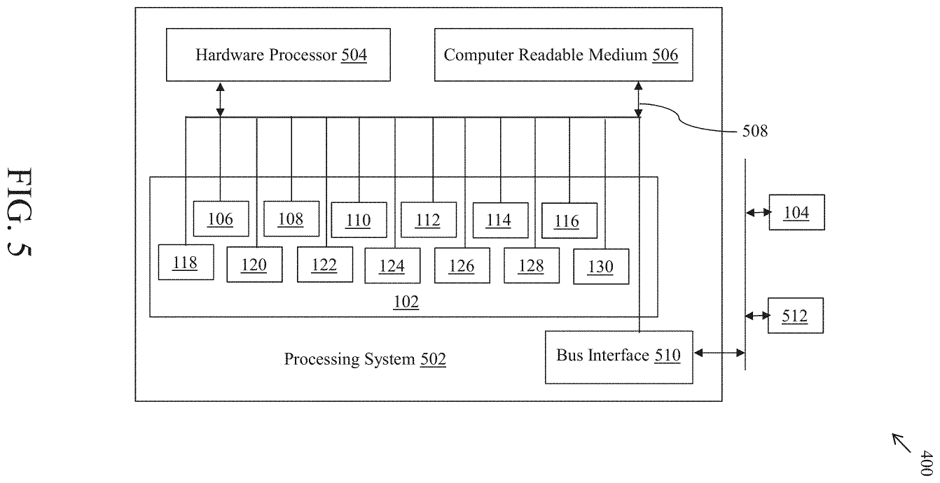

FIGS. 3A and 3B, collectively, depict flowcharts illustrating exemplary operations for generating a novel molecular structure using a protein structure, in accordance with a first exemplary embodiment of the disclosure. Flowcharts 300A and 300B of FIGS. 3A and 3B, respectively, are described in conjunction with FIG. 1 and FIGS. 2A to 2F. Further, the flowcharts 300A and 300B are described in conjunction with an inferential pipeline 400, depicted in FIG. 4.

At step 302, the protein voxel representation 203 of the given protein structure 201 may be generated that comprises a multichannel 3D grid. In accordance with an embodiment, the voxel generator 106 may be configured to generate the protein voxel representation 203 of the given protein structure 201. The protein voxel representation 203 comprises a multichannel 3D grid. The multichannel 3D grid may include a plurality of channels that comprises information regarding a plurality of properties of the given protein structure 201. The plurality of channels in the multichannel 3D grid may include a protein channel that corresponds to the shape of the given protein structure 201, another channel that corresponds to an electrostatic potential of the given protein structure 201 and remaining channels that correspond to two variations of Lennard-Jones potential for a plurality of atom types. The atom types may include a hydrophobic atom, an aromatic atom, a hydrogen bond acceptor, a hydrogen bond donor, a positive ionizable atom, a negative ionizable atom, a metal atom type, and an excluded volume atom.

At step 304, the plurality of channels may be augmented to resolve sparsity in the protein voxel representation 203. In accordance with an embodiment, the augmentation module 108 may be configured to augment the plurality of channels to resolve sparsity in the protein voxel representation 203. The sparsity may correspond to zero values of one or more voxels in the protein voxel representation 203.

At step 306, a cavity region may be detected in the protein voxel representation 203 of the given protein structure 201 based on a combination of rule-based detection and a deep learning-based model. In accordance with an embodiment, the hybrid cavity detector 214 in the cavity detector 110 may be configured to detect a cavity region in the protein voxel representation 203 of the given protein structure 201 based on a combination of rule-based detection performed by the rule-based detector 210 and a deep learning-based model performed by the DL-based detector 212.

At step 308, the higher resolution voxel representation 217 of the detected cavity region may be generated based on the upscaling of the regional voxel detected cavity region using an AI upscaling operation. In accordance with an embodiment, the upscaling module 216 in the cavity detector 110 may be configured to generate the higher resolution voxel representation 217 of the detected cavity region based on the upscaling of the regional voxel detected cavity region using the AI upscaling operation.

At step 310, voxel values in the generated higher resolution voxel representation 217 may be inverted. In accordance with an embodiment, the upscaling module 216 in the cavity detector 110 may be configured to invert voxel values in the generated higher resolution voxel representation 217. The inversion of the voxels may correspond to converting ones to zeros and zeros to ones.

At step 312, the cavity voxel representation 219 of the detected cavity region may be generated based on at least an upscaling of a regional voxel of the detected cavity region. In accordance with an embodiment, the upscaling module 216 in the cavity detector 110 may be configured to generate the cavity voxel representation 219 of the detected cavity region based on at least an upscaling of a regional voxel of the detected cavity region. Thus, the generation of the cavity voxel representation 219 of the cavity region is further based on the inversion of the voxel values in the generated higher resolution voxel representation 217.

At step 314, the ligand voxel representation 221 of a ligand structure may be generated based on at least the cavity voxel representation 219 of the detected cavity region. In accordance with an embodiment, the 3D GAN 112 may be configured to generate the ligand voxel representation 221 of the ligand structure based on at least the cavity voxel representation 219 of the detected cavity region. In accordance with the first exemplary embodiment of the disclosure, the control passes to step 316 for the determination of the 3D voxel descriptor 237. In accordance with the second exemplary embodiment of the disclosure, the control passes to step 322 in flowchart 300B of FIG. 3B, for the prediction of the actual complex voxel representation 235.

At step 316, the 3D voxel descriptor 237 may be determined for a protein-ligand complex based on the protein voxel representation 203 of the given protein structure 201 and the ligand voxel representation 221 of the ligand structure. In accordance with an embodiment, the complex voxel generator 234 may be configured to determine the 3D voxel descriptor 237 for a protein-ligand complex based on the protein voxel representation 203 of the given protein structure 201 and the ligand voxel representation 221 of the ligand structure, as shown in FIG. 4.

At step 318, the rich 3D embedding vector 249 may be generated using the determined 3D voxel descriptor 237. In accordance with an embodiment, the complex voxel generator 234 may be configured to generate the rich 3D embedding vector 249 using the determined 3D voxel descriptor 237. The rich 3D embedding vector 249 may correspond to a single vector of predetermined length representing a protein sequence of the given protein structure 201. The rich 3D embedding vector 249 may be used to predict one or more properties that include at least affinity score and potential bioactivity (such as K_{D}, IC50 (Inhibitory concentration 50)) of the novel molecular structure 251. In accordance with an embodiment, the one or more properties are transmitted back to the 3D GAN 112 to further improve the generation of the ligand voxel representation 221 of the ligand structure.

At step 320, SMILES of the novel molecular structure 251 may be generated using the rich 3D embedding vector, which is based on the determined 3D voxel descriptor 237. In accordance with an embodiment, the 3D caption generator network 116 may be configured to generate the SMILES of the novel molecular structure 251 using the rich 3D embedding vector 249, which is based on the determined 3D voxel descriptor 237. The generated SMILES may correspond to a line notation for describing the novel molecular structure 251 generated based on the multichannel 3D grid of the given protein structure 201. In accordance with an embodiment, the novel molecular structure 251 may be described using short American Standard Code for Information Interchange (ASCII) strings.

FIG. 3C depicts another flowchart illustrating exemplary operations for generating a novel molecular structure using a protein structure, in accordance with a second embodiment of the disclosure. Flowchart 300C of FIG. 3C is described in conjunction with FIG. 1, FIGS. 2A to 2F and FIGS. 3A and 3B.

At step 322, when control is received from step 314 in flowchart 300A of FIG. 3A, the actual complex voxel representation 235 of the given protein structure 201 may be predicted based on a trained deep learning model. In accordance with an embodiment, the CNN model 232 may be configured to predict the actual complex voxel representation 235 of the given protein structure 201 based on a trained deep learning model. In accordance with the second exemplary embodiment of the disclosure, the control passes to step 324 for the generation of the multichannel convolved voxel representation 233 of the ligand structure. In accordance with the third exemplary embodiment of the disclosure, the control passes to step 326 in flowchart 300D of FIG. 3D, for training the 3D VAE 118.

At step 324, a multichannel convolved voxel representation 233 of the ligand structure may be generated based on convolution of the protein voxel representation 203 and the multi-orientated ligand voxel representation 231. In accordance with an embodiment, the multichannel voxel generator 230 may be configured to generate the multichannel convolved voxel representation 233 of the ligand structure based on convolution of the protein voxel representation 203 and the multi-orientated ligand voxel representation 231. The multichannel convolved voxel representation 233 may include a set of channels that comprises information regarding different random orientations of the ligand structure. The control may pass back to step 316 in flowchart 300A of FIG. 3A to return the generated multichannel convolved voxel representation 233 to the complex voxel generator 234 for the generation of the 3D voxel descriptor 237.

FIG. 3D depicts another flowchart illustrating exemplary operations for generating a novel molecular structure using a protein structure, in accordance with a third embodiment of the disclosure. Flowchart 300D of FIG. 3D is described in conjunction with FIG. 1, FIGS. 2A to 2F, and FIGS. 3A to 3C.

At step 326, when control is received from step 322 in flowchart 300C of FIG. 3C, the 3D VAE 118 may be trained using the first rich 3D embedding vector 241 based on the actual complex voxel representation 235 of the given protein structure 201. In accordance with an embodiment, the processor 120 may be configured to train the 3D VAE 118 using the first rich 3D embedding vector 241 based on the actual complex voxel representation 235 of the given protein structure 201.

At step 328, a plurality of reward functions may be optimized using reinforcement learning on top of the VAE. In accordance with an embodiment, the reinforcement learning module 118a may be configured to optimize the plurality of reward functions include affinity, novelty, and absorption, distribution, metabolism, excretion, and toxicity (ADMET). The optimization may be performed by the affinity predictor 248, the novelty predictor 250, and the ADMET predictor 252.

At step 330, the new 3D voxel descriptor 247 may be generated for the protein-ligand complex with intended properties based on the optimized plurality of reward functions. In accordance with an embodiment, the new complex voxel generator 244 may be configured to generate the new 3D voxel descriptor 247 for the protein-ligand complex with intended properties based on the optimized plurality of reward functions.

At step 332, a new SMILES may be generated based on the new 3D voxel descriptor 247. In accordance with an embodiment, the 3D caption generator network 116 may be configured to generate the new SMILES of the novel molecular structure 251 based on the new 3D voxel descriptor 247.

Thus, the disclosed method generates novel molecular structures with desired properties. The disclosed method may find its application in various domains, such as drug discovery. In drug discovery, the disclosed method may be leveraged to generate drug molecules (that satisfies several criteria, such as binding to the specific protein target, suitable absorption by the body, and non-toxicity) by providing appropriate objective (reward) functions, using appropriate input datasets, using pre- and post-processing filters and so on. Other potential applications may find a place in, for example, the paint industry, lubricant industry, and the like.

FIG. 5 is a conceptual diagram illustrating an example of a hardware implementation for a system employing a processing system for generating a novel molecular structure using a protein structure, in accordance with an exemplary embodiment of the disclosure. Referring to FIG. 5, the hardware implementation is shown by a representation 500 for the computing device 102 that employs a processing system 502 for generating a novel molecular structure using a protein structure, as described herein.

In some examples, the processing system 502 may comprise one or more instances of a hardware processor 504, a non-transitory computer-readable medium 506, a bus 508, a bus interface 510, and a transceiver 512. FIG. 5 further illustrates the voxel generator 106, the augmentation module 108, the cavity detector 110, the 3D GAN 112, the convolved voxel generator 114, the 3D caption generator network 116, the 3D VAE 118, the processor 120, the memory 122, the storage device 124, the wireless transceiver 126, and the user interface 128, as described in detail in FIG. 1.

The hardware processor 504, such as the processor 120, may be configured to manage the bus 508 and general processing, including the execution of a set of instructions stored on the computer-readable medium 506. The set of instructions, when executed by the hardware processor 504, causes the computing device 102 to execute the various functions described herein for any particular apparatus. The hardware processor 504 may be implemented based on a number of processor technologies known in the art. Examples of the hardware processor 504 may be the RISC processor, ASIC processor, CISC processor, and/or other processors or control circuits.

The non-transitory computer-readable medium 506 may be used for storing data that is manipulated by the hardware processor 504 when executing the set of instructions. The data is stored for short periods or in the presence of power. The computer-readable medium 506 may also be configured to store data for one or more of the voxel generator 106, the augmentation module 108, the cavity detector 110, the 3D GAN 112, the convolved voxel generator 114, the 3D caption generator network 116, and the 3D VAE 118.

The bus 508 may be configured to link together various circuits. In this example, the computing device 102 employing the processing system 502 and the non-transitory computer-readable medium 506 may be implemented with a bus architecture, generally represented by bus 508. The bus 508 may include any number of interconnecting buses and bridges depending on the specific implementation of the computing device 102 and the overall design constraints. The bus interface 510 may be configured to provide an interface between the bus 508 and other circuits, such as the transceiver 512, and external devices, such as the data sources 104.

The transceiver 512 may be configured to provide communication of the computing device 102 with various other apparatus, such as the data sources 104, via a network. The transceiver 512 may communicate via wireless communication with networks, such as the Internet, the Intranet, and/or a wireless network, such as a cellular telephone network, a wireless local area network (WLAN), and/or a metropolitan area network (MAN). The wireless communication may use any of a plurality of communication standards, protocols, and technologies, such as 5th generation mobile network, Global System for Mobile Communications (GSM), Enhanced Data GSM Environment (EDGE), Long Term Evolution (LTE), wideband code division multiple access (W-CDMA), code division multiple access (CDMA), time division multiple access (TDMA), Bluetooth, Wireless Fidelity (Wi-Fi) (such as IEEE 802.11a, IEEE 802.11b, IEEE 802.11g and/or IEEE 802.11n), voice over Internet Protocol (VoIP), and/or Wi-MAX.

It should be recognized that, in some embodiments of the disclosure, one or more components of FIG. 5 may include software whose corresponding code may be executed by at least one processor across multiple processing environments. For example, the voxel generator 106, the augmentation module 108, the cavity detector 110, the 3D GAN 112, the convolved voxel generator 114, the 3D caption generator network 116, the 3D VAE 118, and the processor 120 may include software that may be executed across a single or multiple processing environments.

In an aspect of the disclosure, the hardware processor 504, the non-transitory computer-readable medium 506, or a combination of both may be configured or otherwise specially programmed to execute the operations or functionality of the voxel generator 106, the augmentation module 108, the cavity detector 110, the 3D GAN 112, the convolved voxel generator 114, the 3D caption generator network 116, the 3D VAE 118, the processor 120, the memory 122, the storage device 124, the wireless transceiver 126, and the user interface 128, or various other components described herein, as described with respect to FIGS. 1 to 4.

Various embodiments of the disclosure comprise the computing device 102 that may be configured to generate a novel molecular structure using a protein structure. The computing device 102 may comprise, for example, the voxel generator 106, the augmentation module 108, the cavity detector 110, the 3D GAN 112, the convolved voxel generator 114, the 3D caption generator network 116, the 3D VAE 118, the processor 120, the memory 122, the storage device 124, the wireless transceiver 126, and the user interface 128. One or more processors, such as the voxel generator 106, in the computing device 102 may be configured to generate a protein voxel representation, such as the protein voxel representation 203 of a protein structure, such as the given protein structure 201. The protein voxel representation 203 may comprise a multichannel 3D grid. The multichannel 3D grid may include a plurality of channels that comprises information regarding a plurality of properties of the given protein structure 201. The one or more processors, such as the cavity detector 110, may be configured to detect a cavity region in the protein voxel representation 203 of the given protein structure 201 based on a combination of the rule-based detection performed by the rule-based detector 210 and a deep learning-based model performed by the DL-based detector 212. The one or more processors, such as the upscaling module 216, may be configured to generate a cavity voxel representation, such as the cavity voxel representation 219 of the detected cavity region based on at least an upscaling of a regional voxel of the detected cavity region. The one or more processors, such as the 3D GAN 112, may be configured to generate a ligand voxel representation, such as the ligand voxel representation 221 of a ligand structure based on at least the cavity voxel representation 219 of the detected cavity region. The one or more processors, such as the complex voxel generator 234, may be configured to determine a 3D voxel descriptor, such as the 3D voxel descriptor 237, for a protein-ligand complex based on the protein voxel representation 203 of the given protein structure 201 and the ligand voxel representation 221 of the ligand structure. The one or more processors, such as the 3D caption generator network 116, may be configured to generate SMILES of a novel molecular structure, such as the novel molecular structure 251, using a rich 3D embedding vector, such as the rich 3D embedding vector 249, which is based on the determined 3D voxel descriptor 237.

Various embodiments of the disclosure may provide a non-transitory computer-readable medium having stored thereon; computer-implemented instruction that when executed by a processor causes the computing device 102 to generate a novel molecular structure using a protein structure. The computing device 102 may execute operations comprising generating the protein voxel representation 203 of the given protein structure 201 that comprises a multichannel 3D grid. The multichannel 3D grid includes a plurality of channels that comprises information regarding a plurality of properties of the given protein structure 201. The computing device 102 may execute further operations comprising detecting a cavity region in the protein voxel representation 203 of the given protein structure 201 based on a combination of rule-based detection and a deep learning-based model. The computing device 102 may execute further operations comprising generating the cavity voxel representation 219 of the detected cavity region based on at least an upscaling of a regional voxel of the detected cavity region. The computing device 102 may execute further operations comprising generating the ligand voxel representation 221 of a ligand structure based on at least the cavity voxel representation 219 of the detected cavity region. The computing device 102 may execute further operations comprising determining the 3D voxel descriptor 237 for a protein-ligand complex based on the protein voxel representation 203 of the given protein structure 201 and the ligand voxel representation 221 of the ligand structure. The computing device 102 may execute further operations comprising generating SMILES of the novel molecular structure 251 using the rich 3D embedding vector 249, which is based on the determined 3D voxel descriptor 237.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether the performance of the function is disabled or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application-specific integrated circuits (ASICs)), by program instructions being executed by one or more processors, or by a combination of both. Additionally, these sequences of actions described herein can be considered to be embodied entirely within any non-transitory form of a computer-readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for generating a novel molecular structure using a protein structure.

The present disclosure may also be embedded in a computer program product, which comprises all the features enabling the implementation of the methods described herein, and which when loaded in a computer system, is able to carry out these methods. The computer program in the present context means any expression, in any language, code, or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences, and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps, and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A system, comprising:
one or more processors in a computing device, the one or more processors are configured to:
generate a protein voxel representation of a protein structure that comprises a multichannel three-dimensional (3D) grid, wherein the multichannel 3D grid includes a plurality of channels that comprises information regarding a plurality of properties of the protein structure;
detect a cavity region in the protein voxel representation of the protein structure based on a combination of rule-based detection and a deep learning-based model;
generate a cavity voxel representation of the detected cavity region based on at least an upscaling of a regional voxel of the detected cavity region;
generate a ligand voxel representation of a ligand structure based on at least the cavity voxel representation of the detected cavity region;
determine a 3D voxel descriptor for a protein-ligand complex based on the protein voxel representation of the protein structure and the ligand voxel representation of the ligand structure; and
generate a simplified molecular-input line-entry system (SMILES) of a novel molecular structure using a rich 3D embedding vector, which is based on the determined 3D voxel descriptor.

2. The system according to claim 1, wherein the plurality of channels in the multichannel 3D grid includes a protein channel that corresponds to the shape of the protein structure, another channel that corresponds to an electrostatic potential of the protein structure and remaining channels that correspond to two variations of Lennard-Jones potential for a plurality of atom types,
wherein the atom types include a hydrophobic atom, an aromatic atom, a hydrogen bond acceptor, a hydrogen bond donor, a positive ionizable atom, a negative ionizable atom, a metal atom type, and an excluded volume atom.

3. The system according to claim 1, wherein the one or more processors are further configured to augment the plurality of channels to resolve sparsity in the protein voxel representation,
wherein the sparsity corresponds to zero values of one or more voxels in the protein voxel representation.

4. The system according to claim 1, wherein, for the generation of the cavity voxel representation of the detected cavity region, the one or more processors are further configured to:
generate a higher resolution voxel representation of the detected cavity region based on the upscaling of the regional voxel detected cavity region using an artificial intelligence (AI) upscaling operation; and
invert voxel values in the generated higher resolution voxel representation,
wherein the generation of the cavity voxel representation of the cavity region is further based on the inversion of the voxel values in the generated higher resolution voxel representation.

5. The system according to claim 1, wherein, for the determination of the 3D voxel descriptor for a protein-ligand complex, the one or more processors are further configured to:
generate a multichannel convolved voxel representation of the ligand structure based on convolution of the protein voxel representation and the ligand voxel representation,
wherein the multichannel convolved voxel representation includes a set of channels that comprises information regarding different random orientations of the ligand structure; and
predict an actual complex voxel representation of the protein structure based on a trained deep learning model,
wherein the determination of the 3D voxel descriptor for the protein-ligand complex is based on the multichannel convolved voxel representation of the ligand structure and the actual complex voxel representation of the protein structure.

6. The system according to claim 5, wherein the one or more processors are further configured to:
train a variational auto encoder (VAE) using another rich 3D embedding vector based on the actual complex voxel representation of the protein structure;
optimize a plurality of reward functions using a reinforcement learning module on top of the VAE; and
generate a new 3D voxel descriptor for the protein-ligand complex with intended properties based on the optimized plurality of reward functions.

7. The system according to claim 6, the one or more processors are further configured to generate a new SMILES based on the new 3D voxel descriptor.

8. The system according to claim 6, wherein the plurality of reward functions include affinity, novelty, and absorption, distribution, metabolism, excretion, and toxicity (ADMET).

9. The system according to claim 1, wherein the generated SMILES corresponds to a line notation for describing the novel molecular structure generated based on the multichannel 3D grid of the protein structure,
wherein the novel molecular structure is described using short American Standard Code for Information Interchange (ASCII) strings.

10. The system according to claim 1, wherein the one or more processors are further configured to generate the rich 3D embedding vector using the determined 3D voxel descriptor,
wherein the rich 3D embedding vector corresponds to a single vector of predetermined length representing a protein sequence of the protein structure,
wherein the rich 3D embedding vector is used to predict one or more properties that include at least affinity score and potential bioactivity of the novel molecular structure.

11. A method, comprising:
generating, by a processor, a protein voxel representation of a protein structure that comprises a multichannel three dimensional (3D) grid,
wherein the multichannel 3D grid includes a plurality of channels that comprises information regarding a plurality of properties of the protein structure;
detecting, by the processor, a cavity region in the protein voxel representation of the protein structure based on a combination of rule-based detection and a deep learning based model;
generating, by the processor, a cavity voxel representation of the detected cavity region based on at least an upscaling of a regional voxel of the detected cavity region;
generating, by the processor, a ligand voxel representation of a ligand structure based on at least the cavity voxel representation of the detected cavity region;
determining, by the processor, a 3D voxel descriptor for a protein-ligand complex based on the protein voxel representation of the protein structure and the ligand voxel representation of the ligand structure; and
generating, by the processor, a simplified molecular-input line-entry system (SMILES) of a novel molecular structure using a rich 3D embedding vector, which is based on the determined 3D voxel descriptor.

12. The method according to claim 11, wherein the plurality of channels in the multichannel 3D grid includes a protein channel that corresponds to shape of the protein structure, another channel that corresponds to an electrostatic potential of the protein structure, and remaining channels that correspond to two variations of Lennard-Jones potential for a plurality of atom types, and
wherein the atom types include a hydrophobic atom, an aromatic atom, a hydrogen bond acceptor, a hydrogen bond donor, a positive ionizable atom, a negative ionizable atom, a metal atom type, and an excluded volume atom.

13. The method according to claim 11, further comprising augmenting, by the processor, the plurality of channels to resolve sparsity in the protein voxel representation,
wherein the sparsity corresponds to zero values of one or more voxels in the protein voxel representation.

14. The method according to claim 11, wherein, for the generation of the cavity voxel representation of the detected cavity region, the method further comprising:
generating, by the processor, a higher resolution voxel representation of the detected cavity region based on the upscaling of the regional voxel detected cavity region using an artificial intelligence (AI) upscaling operation; and
inverting, by the processor, voxel values in the generated higher resolution voxel representation,
wherein the generation of the cavity voxel representation of the cavity region is further based on the inversion of the voxel values in the generated higher resolution voxel representation.

15. The method according to claim 11, wherein, for the determination of the 3D voxel descriptor for a protein-ligand complex, the method further comprising:
generating, by the processor, a multichannel convolved voxel representation of the ligand structure based on convolution of the protein voxel representation and the ligand voxel representation,
wherein the multichannel convolved voxel representation includes a set of channels that comprises information regarding different random orientations of the ligand structure; and
predicting, by the processor, an actual complex voxel representation of the protein structure based on a trained deep learning model,
wherein the determination of the 3D voxel descriptor for the protein-ligand complex is based on the multichannel convolved voxel representation of the ligand structure and the actual complex voxel representation of the protein structure.

16. The method according to claim 15, further comprising:
training, by the processor, a variational auto encoder (VAE) using another rich 3D embedding vector based on the actual complex voxel representation of the protein structure;
optimizing, by the processor, a plurality of reward functions using a reinforcement learning module on top of the VAE; and
generating, by the processor, a new 3D voxel descriptor for the protein-ligand complex with intended properties based on the optimized plurality of reward functions.

17. The method according to claim 16, further comprising generating, by the processor, a new SMILES based on the new 3D voxel descriptor,
wherein the plurality of reward functions include affinity, novelty, and absorption, distribution, metabolism, excretion, and toxicity (ADMET).

18. The method according to claim 11, wherein the generated SMILES corresponds to a line notation for describing the novel molecular structure generated based on the multichannel 3D grid of the protein structure, and
wherein the novel molecular structure is described using short American Standard Code for Information Interchange (ASCII) strings.

19. The method according to claim 11, further comprising generating, by the processor, the rich 3D embedding vector using the determined 3D voxel descriptor,
wherein the rich 3D embedding vector corresponds to a single vector of predetermined length representing a protein sequence of the protein structure, and
wherein the rich 3D embedding vector is used to predict intended properties that include at least affinity score and potential bioactivity of the novel molecular structure.

20. A non-transitory computer-readable medium, having stored thereon, computer-executable code, which when executed by a processor, cause the processor to execute operations, the operations comprising:
generating a protein voxel representation of a protein structure that comprises a multichannel three dimensional (3D) grid,
wherein the multichannel 3D grid includes a plurality of channels that comprises information regarding a plurality of properties of the protein structure;
detecting a cavity region in the protein voxel representation of the protein structure based on a combination of rule-based detection and a deep learning based model;
generating a cavity voxel representation of the detected cavity region based on at least an upscaling of a regional voxel of the detected cavity region;
generating a ligand voxel representation of a ligand structure based on at least the cavity voxel representation of the detected cavity region;
determining a 3D voxel descriptor for a protein-ligand complex based on the protein voxel representation of the protein structure and the ligand voxel representation of the ligand structure; and
generating a simplified molecular-input line-entry system (SMILES) of a novel molecular structure using a rich 3D embedding vector, which is based on the determined 3D voxel descriptor.
